(19) United States
(12) Patent Application Publication
(10) Pub. No.: US 2010/0063333 A1
(43) Pub. Date: Mar. 11, 2010

(54) Catalytic Hydrochlorination System and Process for Manufacturing Vinyl Chloride from Acetylene and Hydrogen Chloride in the Presence of this Catalytic System

(75) Inventors: Andre Petitjean, Brussels (BE); Michel Strebelle, Brussels (BE); Andre Devos, La Hulpe (BE)

Correspondence Address: Solvay;c/o B. Ortego - IAM-NAFTA, 3333 Richmond Avenue, Houston, TX, 77098-3099, US

(73) Assignee: SOLVAY (SOCIETE ANONYME), Brussels (BE)

(21) Appl. No.: 12/520,497

(22) PCT Filed: Dec. 20, 2007
(86) PCT No.: PCT/EP2007/064280

§ 371 Date: Jun. 19, 2009

(30) Foreign Application Priority Data
Dec. 22, 2006 (FR) 0611356

(51) Int. Cl. C07C 17/08; B01J 27/24; B01J 31/00; B01J 31/02

(52) U.S. Cl. 570/233; 502/200; 502/150; 502/162; 502/167

(57) ABSTRACT

Catalytic hydrochlorination system comprising at least one amine hydrochloride and at least one group VIII metal compound chosen from the group composed of mixtures of a platinum (IV) compound with tin (II) chloride, mixtures of a platinum (II) compound with triphenylphosphine oxide and mixtures of a palladium (II) compound with triphenylphosphine. This catalytic system is suitable for preparing vinyl chloride by reaction of acetylene with hydrogen chloride.

The present invention relates to a catalytic hydrochlorination system based on a group VIII metal compound and a process for manufacturing vinyl chloride by hydrochlorination of acetylene in the presence of such a catalytic system.

The manufacture of vinyl chloride by reaction between acetylene and hydrogen chloride is conventionally carried out in the gas phase, in a fixed-bed reactor, in the presence of a heterogeneous solid catalyst based on mercury chloride on a support. Mainly for reasons of toxicity, there is currently an increasing interest in catalytic systems that are free of mercury compounds. Various catalysts intended to replace the current catalysts in gas-phase processes have been developed. For example, unexamined Japanese Patent Application 52/136104 describes a process of hydrochlorinating acetylene in the gas phase in the presence of a fixed catalyst bed composed of noble metal halides deposited on activated carbon. To date however, the lifetime of such alternative catalysts intended for gas-phase processes remains much shorter than that of catalysts based on mercury compounds.

Furthermore, in the literature there are some examples of hydrochlorinating acetylene in the presence of a liquid catalytic medium. German Patent 709.000 describes a process for preparing vinyl halides by bringing acetylene into contact, at high temperature, with a molten mass of hydrohalide salts of organic bases containing a standard catalyst. Aliphatic, aromatic or heterocyclic amines and mixtures thereof are envisaged as organic bases. In Example 1, vinyl chloride is obtained by dispersion of hydrogen chloride and acetylene in a mixture composed of 350 parts by volume of pyridine, 350 parts by volume of diethylamine and 100 parts by weight of mercury chloride, kept at 220-225° C. Inventor's certificate SU 237116 describes the use of an aqueous acid solution containing 46 wt % of cuprous chloride and from 14 to 16 wt % of a methylamine, dimethylamine or trimethylamine hydrochloride. Patent Application EP-A-0 340 416 discloses a process for preparing vinyl chloride by reaction of acetylene with hydrogen chloride in the presence of a palladium compound as catalyst in a solvent composed of an aliphatic or cycloaliphatic amide, at a temperature above ambient temperature. Although it allows high yields to be obtained, this process has, however, some significant drawbacks: it has emerged that, under the reaction conditions, the liquid catalyst system gradually degrades, forming blackish products of carbonaceous appearance. In addition, in the presence of hydrogen chloride, the amide is converted to a hydrochloride, the melting point of which is generally much higher than ambient temperature. N-Methylpyrrolidone hydrochloride, for example, is only liquid above 80° C. In practice, this may cause serious implementation problems, problems linked to agglomeration of the catalytic medium during reactor shutdowns or blocking of the lines at the coldest points of the installation. The entire reactor and also the lines in which the reaction medium flows must then be continuously kept at a temperature above the melting point of the hydrochloride.

These various problems seemed to have been solved thanks to the catalytic hydrochlorination systems described in Patent Applications EP 0 519 548-A1 and EP 0 525 843-A1 and that comprise at least one group VIII metal compound and either an amine hydrochloride, the melting point of which is less than or equal to 25° C., or a fatty amine hydrochloride comprising more than 8 carbon atoms, the melting point of which is above 25° C. and an organic solvent chosen from aliphatic, cycloaliphatic and aromatic hydrocarbons and mixtures thereof. Nevertheless, the catalyst systems that are described therein, especially those of which the group VIII metal compound is platinum (II) chloride or palladium (II) chloride, are not completely satisfactory when considering the performances that they enable to be achieved in terms of productivity of the vinyl chloride produced by hydrochlorination of acetylene.

Consequently, one subject of the invention is a catalytic hydrochlorination system free of mercury compounds that is easy to implement due to remaining liquid at ambient temperature and that is better performing than the predecessors. Another subject of the invention is a process for synthesizing vinyl chloride by hydrochlorination of acetylene in the presence of such a catalytic system which does not degrade under the reaction conditions and which makes it possible to achieve a better productivity of the vinyl chloride produced. Unlike systems based on mercury compounds, the catalytic system according to the invention furthermore has the advantage of not having toxicity problems linked to these compounds and of avoiding the vaporization of metal salts in the installation.

The invention therefore relates to a catalytic hydrochlorination system, more particularly a catalytic system for the hydrochlorination of acetylene. This catalytic system comprises at least one amine hydrochloride and at least one group VIII metal compound chosen from the group composed of mixtures of a platinum (IV) compound with tin (II) chloride, mixtures of a platinum (II) compound with triphenylphosphine oxide and mixtures of a palladium (II) compound with triphenylphosphine.

The expression “at least one group VIII metal compound” is understood to mean that the catalytic hydrochlorination system may comprise one or more than one thereof. Preferably, it contains only one thereof.

Any platinum (IV), platinum (II) or palladium (II) compound may be used in the catalytic system of the present invention as long as it can be converted to a chloride in the presence of hydrogen chloride during the preparation of the catalytic system. Thus, nitrates, acetates, carbonates or oxides of platinum (IV), platinum (II) or palladium (II) may be used. Chloride-based compounds of these metals are nevertheless preferred.

Among the chloride-based compounds of platinum (IV), mention may be made of platinum (IV) chloride and hexachloroplatinic acid or its salts, for example Na₂PtCl₆, K₂PtCl₆ or Li₂PtCl₆.

Among the chloride-based compounds of platinum (II), mention may be made of platinum (II) chloride and the platinochlorides of alkali metals or of alkaline-earth metals, such as for example Na₂(PtCl₄), K₂(PtCl₄), Li₂(PtCl₄) and (NH₄)₂(PtCl₄).

Among the chloride-based compounds of palladium (II), mention may be made of palladium (II) chloride and the palladochlorides of alkali metals or of alkaline-earth metals, such as for example Na₂(PdCl₄), K₂(PdCl₄), Li₂(PdCl₄) and (NH₄)₂(PdCl₄).

Particularly preferably, platinum (IV) chloride, platinum (II) chloride and palladium (II) chloride are chosen as compounds of platinum (IV), platinum (II) and palladium (II) respectively.

The group VIII metal compound is thus particularly preferably chosen from the group composed of the mixture of platinum (IV) chloride with tin (II) chloride, the mixture of platinum (II) chloride with triphenylphosphine oxide and the mixture of palladium (II) chloride with triphenylphosphine. The latter two mixtures mentioned receive, most particularly preferably, special attention.

The expression “at least one amine hydrochloride” is understood to mean that the catalytic hydrochlorination system may comprise one or more than one thereof. Preferably, it contains only one thereof.

The molar ratio of the tin (II) chloride, the triphenylphosphine oxide or the triphenylphosphine to the group VIII metal of the catalytic system according to the invention is advantageously at least 0.5, preferably at least 1. This molar ratio is advantageously at most 5, preferably at most 2. A molar ratio between 0.5 and 2 is particularly preferred.

According to a first preferred variant, the amine hydrochloride is advantageously chosen from the amine hydrochlorides of which the melting point is less than or equal to 25° C.

Amine hydrochlorides of which the melting point is less than or equal to 25° C. are especially amine hydrochlorides having high steric hindrance, such as the amine hydrochlorides corresponding to the following generic formula:

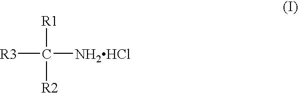

with R1 and R2 representing hydrogen atoms or identical or different alkyl or aryl groups and R3 an alkyl or aryl group, said amine hydrochloride containing from 8 to 30 carbon atoms. Optionally R1 and R3 may together form, by means of carbon atoms connecting them, a ring, for example having 5 or 6 carbon atoms, which may be substituted by alkyl groups. Preferably, R1, R2 and R3 are alkyl groups.

The expression “alkyl group” is understood to mean any linear or branched carbon-based chain, optionally substituted by one or more aryl groups. The expression “aryl group” is understood to mean any aromatic radical optionally substituted by one or more other groups, such as alkyl groups for example.

The total number of carbon atoms in this compound is advantageously at least equal to 8. It is preferably at least equal to 10. The total number of carbon atoms in this compound is advantageously at most equal to 30. It is preferably at most equal to 25.

The expression “amine hydrochloride” is understood to mean one or more amine hydrochlorides, including any mixture of hydrochlorides of several amines, for example, of several isomeric compounds.

Such a mixture of hydrochlorides of several amines may also be used, especially due to its greater availability or its lower cost relative to pure compounds. An example of such an amine hydrochloride comprising a mixture of various compounds corresponding to the formula (I) is obtained by reaction of hydrogen chloride with commercial products such as the tert-alkyl primary amines PRIMENE 81-R and PRIMENE JM-T from Rohm and Haas Co., composed of mixtures of C₁₂-C₁₄ and C₁₈-C₂₂ isomeric amines respectively. In certain circumstances it may also prove advantageous to deliberately mix hydrochlorides of various amines due to the existence of eutectics between these compounds, having a melting point below that of each of the constituents.

Good results have been obtained with a catalytic system comprising a hydrochloride of a tert-alkylamine (R1, R2 and R3 representing alkyl groups) containing from 10 to 25 carbon atoms such as the tert-alkyl primary amines PRIMENE 81-R and PRIMENE JM-T from Rohm and Haas Co. Other amine hydrochlorides that have also given good results are the hydrochlorides of amines in which R1 and R2 are hydrogen atoms and R3 is an aryl or alkyl group, for example polyisopropylbenzylamine hydrochloride and polyethyl-β-phenethylamine hydrochloride. Such amines having high steric hindrance may be easily obtained, for example, starting from the corresponding amines, the aromatic ring of which is not alkylated, for the above compounds, starting respectively from benzylamine and 2-phenylethylamine via protection of the amine functional group by reaction with a carboxylic acid anhydride, conventional alkylation of the aromatic ring of the amide obtained and finally alkaline hydrolysis of the amide functional group.

The catalytic system particularly preferred according to the first variant contains, as amine hydrochloride, a tert-alkylamine hydrochloride, for example that obtained from the tert-alkyl primary amine PRIMENE 81-R.

The content of group VIII metal compound in the catalytic system according to the first preferred variant, expressed in millimoles per litre of amine hydrochloride is advantageously greater than or equal to about 1 mmol/l and less than or equal to about 1000 mmol/l. The content of group VIII metal compounds in the catalytic system according to the first preferred variant is advantageously greater than or equal to about 1 mmol/l, preferably greater than or equal to about 5 mmol/l and particularly preferably greater than or equal to about 10 mmol/l.

The content of group VIII metal compound in the catalytic system is advantageously less than or equal to about 1000 mmol/l, preferably less than or equal to about 500 mmol/l, particularly preferably less than or equal to about 200 mmol/l, more particularly preferably less than or equal to about 100 mmol/l and most particularly preferably less than or equal to about 50 mmol/l. Although it is not essential, it is however preferable that all the group VIII metal compound included in the catalytic system be in dissolved form.

The catalytic system defined above may be used in the liquid phase or be deposited on a solid support such as a silica, alumina or activated carbon, up to the limit of the pore volume of the support. When it is used in the liquid phase, the catalytic system is preferably diluted by an organic solvent. The choice of the nature of the organic solvent then included in the catalytic system according to the invention especially depends on the requirement that it be inert with respect to the reactants under the reaction conditions, that it be miscible with the amine hydrochloride and on the desire that it form with this hydrochloride a medium, the viscosity of which is lower than that of the hydrochloride alone. Furthermore, for reasons of safety and ease of use, preference is given to organic solvents that are not very volatile. The choice of organic solvent is also influenced by its acetylene absorption capacity. The solvents that satisfy the various criteria explained above are chosen from aliphatic, cycloaliphatic and aromatic hydrocarbons and mixtures thereof, for example C₇ to C₁₅ paraffins and alkylbenzenes, especially xylenes, propylbenzenes, butylbenzenes and methylethylbenzenes. For economic reasons, the solvent used is preferably chosen from commercial products composed of mixtures of aliphatic hydrocarbons such as the solvent ISOPAR from Esso or the solvent SHELLSOL K from Shell or mixtures of aromatic compounds such as the solvent SOLVESSO from Esso or the solvent SHELLSOL AB from Shell.

Solvents that have given good results are saturated aliphatic solvents, such as the solvent SHELLSOL K composed of cuts having a boiling point between about 190° C. and about 250° C.

Other solvents that can be envisaged based on the various criteria given above are certain heavy halogenated compounds, such as haloalkanes, halobenzenes and other halogenated derivatives of aromatic compounds.

When the catalytic system is used in the liquid phase, the catalytic system that is most particularly preferred according to the first preferred variant contains, as amine hydrochloride, a tert-alkylamine hydrochloride, for example that obtained from the tent-alkyl primary amine PRIMENE 81-R and, as organic solvent, an aliphatic solvent such as the solvent SHELLSOL K.

When the catalytic system is used in the liquid phase and when it is diluted by an organic solvent, the weight ratio of the solvent to the amine hydrochloride is advantageously greater than or equal to about 0.01. Preferably, this ratio is greater than or equal to about 0.05. Under particularly preferred conditions, it is greater than or equal to about 0.2. This ratio is advantageously less than or equal to about 5. Preferably, it is less than or equal to about 3. Under particularly preferred conditions, it is less than or equal to about 2.

Generally, the catalytic system is prepared by dissolving or dispersing the desired amount of group VIII metal compound in the amine or in the amine/organic solvent mixture, then saturating this solution with hydrogen chloride that gives rise to the formation of the amine hydrochloride. However, it is also possible to first saturate the amine or the amine/organic solvent mixture with hydrogen chloride in order to form the amine hydrochloride, then to next introduce the group VIII metal compound into the amine hydrochloride or into the mixture of the latter with the organic solvent. Usually, the amount of group VIII metal compound used is such that, in the catalytic system, the entire group VIII metal compound is in dissolved form. By way of indication, the solubility of platinum (II) chloride in the mixture of equal parts by weight of amine hydrochloride PRIMENE 81-R and of solvent SHELLSOL K exceeds 1 mol/l. However, it is also possible to use a group VIII metal compound in an amount or of a nature such that at least one fraction of this compound is present in the catalytic system in the form of a dispersed solid, without prejudicing the invention.

According to a second variant, the catalytic system is such that the amine hydrochloride is advantageously chosen from fatty amine hydrochlorides of which the melting point is greater than 25° C. and that it comprises, in addition, an organic solvent.

The expression “fatty amine” is understood to mean any amine or mixtures of amines containing a large number of carbon atoms, preferably more than 8 carbon atoms, having a molecular structure that is slightly branched or unbranched. Particularly preferred amines are those that contain from 10 to 20 carbon atoms. This slightly branched or unbranched molecular structure allows easy crystallization of the hydrochloride formed by reaction of the fatty amine with hydrogen chloride and explains the high melting points of the hydrochlorides of these compounds. Amines that correspond to the definition of a fatty amine above are, for example, decylamine, undecylamine, dodecylamine and 3-methyldodecylamine.

Good results have been obtained with a catalytic system comprising dodecylamine hydrochloride.

The choice of the nature of the organic solvent included in the catalytic system according to the invention especially depends on the requirement that it be inert with respect to the reactants under the reaction conditions, that it be miscible with the fatty amine hydrochloride at the reaction temperature and that it be capable of solubilizing the latter at a temperature below its melting point. Furthermore, for reasons of safety and ease of use, preference is given to organic solvents that are not very volatile. The choice of organic solvent is also influenced by its acetylene absorption capacity. The solvents that satisfy the various criteria explained above are chosen from aliphatic, cycloaliphatic and aromatic hydrocarbons and mixtures thereof, such as defined previously for the first preferred variant of the catalytic system according to the invention.

The catalytic system that is most particularly preferred according to the second variant contains, as amine hydrochloride, dodecylamine hydrochloride and, as organic solvent, an aliphatic solvent such as the solvent SHELLSOL K.

The weight ratio of the organic solvent to the fatty amine hydrochloride advantageously varies from about 0.1 to about 20 and the content of group VIII metal compound expressed in millimoles per litre of catalytic system is advantageously greater than or equal to about 1 mmol/l and less than or equal to about 1000 mmol/l.

The weight ratio of the organic solvent to the fatty amine hydrochloride is advantageously greater than or equal to about 0.1. Preferably, this ratio is greater than or equal to about 0.5. Under particularly preferred conditions, it is greater than or equal to about 0.8. Advantageously, this ratio is less than or equal to about 20. Preferably, it is less than or equal to about 10. Under particularly preferred conditions, it is less than or equal to about 8.

The content of group VIII metal compound in the catalytic system according to the second variant, expressed in millimoles per litre of solution of catalytic system, is advantageously greater than or equal to about 1 mmol/l, preferably greater than or equal to about 5 mmol/l and particularly preferably greater than or equal to about 10 mmol/l. The content of group VIII metal compound in the catalytic system according to the second variant is advantageously less than or equal to about 1000 mmol/l, preferably less than or equal to about 500 mmol/l, particularly preferably less than or equal to about 200 mmol/l, more particularly preferably less than or equal to about 100 mmol/l and most particularly preferably less than or equal to about 50 mmol/l.

Although it is not essential, it is however preferable that all the group VIII metal compound included in the catalytic system be in dissolved form. Generally, the catalytic system is prepared by dissolving or dispersing the desired amount of group VIII metal compound in the fatty amine/organic solvent mixture, by heating this solution to a temperature above the melting point of the fatty amine hydrochloride, then by saturating this solution with hydrogen chloride that gives rise to the formation of the fatty amine hydrochloride. However, it is also possible, although less easy in practice, to first saturate the preheated fatty amine/organic solvent mixture with hydrogen chloride in order to form the fatty amine hydrochloride, then to next introduce the group VIII metal compound into the fatty amine hydrochloride or into the mixture of the latter with the organic solvent. Usually, the amount of group VIII metal compound used is such that, in the catalytic system, the entire group VIII metal compound is in dissolved form. However, it is also possible to use a group VIII metal compound in an amount or of a nature such that at least one fraction of this compound is present in the catalytic system in the form of a dispersed solid, without prejudicing the invention.

The invention also relates to a process for manufacturing vinyl chloride by reaction of acetylene with hydrogen chloride (hydrochlorination) in the presence of a catalytic system comprising at least one amine hydrochloride and at least one group VIII metal compound chosen from the group composed of mixtures of a platinum (IV) compound with tin (II) chloride, mixtures of a platinum (II) compound with triphenylphosphine oxide and mixtures of a palladium (II) compound with triphenylphosphine.

The nature and the amounts of the constituents of the catalytic system used in the process according to the invention are those defined above for the catalytic system according to the invention.

According to a first preferred variant, the process according to the invention is advantageously such that the amine hydrochloride is chosen from the amine hydrochlorides of which the melting point is less than or equal to 25° C., such as defined previously for the catalytic system according to the invention.

According to this first preferred variant of the process according to the invention, the catalytic system may be used in the liquid phase. It may also be deposited on a solid support such as a silica, alumina or activated carbon, up to the limit of the pore volume of the support. Preferably, the catalytic system is used in the liquid phase. However, the viscosity of this liquid at the reaction temperature often limits the efficiency of the exchange of matter between the gas phase containing the reactants and the liquid phase in which the hydrochlorination reaction takes place. Therefore, the catalytic system is preferably diluted by an organic solvent such as defined previously for the catalytic system according to the invention.

According to the first preferred variant, the process according to the invention can advantageously be carried out from ambient temperature up to about 220° C. At higher temperatures, the catalytic system has a tendency to degrade rapidly. The preferred reaction temperature, that is to say that offering the best compromise between productivity, yield and stability of the catalytic medium, is greater than or equal to about 40° C. The best results are obtained at temperatures greater than or equal to about 50° C. with a more particular preference for temperatures greater than or equal to about 80° C. and a most particular preference for temperatures greater than or equal to about 120° C.

Preferably, the reaction temperature does not exceed about 200° C. A reaction temperature less than or equal to about 170° C. is particularly preferred.

A reaction temperature of about 40° C. to about 200° C. is most particularly preferred.

According to a second variant, the process according to the invention is advantageously such that the amine hydrochloride is chosen from the fatty amine hydrochlorides of which the melting point is greater than 25° C., such as defined previously for the catalytic system according to the invention, and that the catalytic system comprises, in addition, an organic solvent, such as also defined previously.

According to this second variant of the process according to the invention, the catalytic system is therefore advantageously used in the liquid phase.

According to the second variant, the process according to the invention can advantageously be carried out from ambient temperature up to about 200° C. At higher temperatures, the catalytic system has a tendency to degrade rapidly. Generally, the reaction temperature is such that all the fatty amine hydrochloride is in solution. The preferred reaction temperature, that is to say that offering the best compromise between productivity, yield and stability of the catalytic medium, is greater than or equal to about 40° C. The best results are obtained at temperatures greater than or equal to about 50° C. with a more particular preference for temperatures greater than or equal to about 80° C. and a most particular preference for temperatures greater than or equal to about 120° C.

Preferably, the reaction temperature does not exceed about 180° C. A reaction temperature less than or equal to about 170° C. is particularly preferred.

A reaction temperature of about 40° C. to about 180° C. is most particularly preferred.

The process according to the invention, according to the first preferred variant or according to the second variant, is advantageously carried out at atmospheric pressure or at a slightly higher pressure compatible with the safety regulations for handling acetylene, that is to say, not exceeding about 1.5 bar of partial pressure of acetylene.

The process for manufacturing vinyl chloride by hydrochlorination of acetylene according to the invention, regardless of its variant, is carried out by bringing the gaseous reactants—acetylene and hydrogen chloride—into contact with the catalytic system, in any suitable reactor.

When the catalytic system is used in the liquid phase, the process according to the invention may be carried out conventionally in any equipment promoting gas-liquid exchange, such as a plate column or a flooded packed column. Another embodiment of the process enabling good exchange of matter between the liquid and gas phases consists of the use of a countercurrent reactor, optionally of the sparged packed-bed type, the liquid catalytic system flowing over the packing, countercurrently to the gaseous flow of reactants.

When the catalytic system is deposited on a suitable solid support, it may advantageously replace the mercury catalysts in the current installations that operate with fixed-bed reactors.

In the process according to the invention, regardless of its variant, the molar ratio of the hydrogen chloride to the acetylene introduced into the reactor is advantageously greater than or equal to about 0.5. Preferably, this ratio is greater than or equal to about 0.8. Advantageously, this molar ratio is less than or equal to about 3. Preferably, the molar ratio of the hydrogen chloride to the acetylene introduced into the reactor is less than or equal to about 1.5.

Good results have been obtained when the hydrogen chloride and the acetylene are used in a molar ratio of about 0.5 to about 3.

The acetylene and the hydrogen chloride may be brought into contact in the reactor or, preferably, mixed prior to being introduced into the reactor.

When operating in a liquid medium, for the purpose of increasing the amount of acetylene dissolved in the liquid phase, it is also possible to use a process in which only the acetylene is introduced into the reactor in gaseous form, where it reacts with the hydrogen chloride present in the liquid phase in hydrochloride form, the amine hydrochloride of the catalytic system being regenerated by bringing a liquid loop containing the amine into contact with hydrogen chloride outside of the reactor. The hydrogen chloride can be introduced in any form: dilute gaseous, pure or dissolved in a solvent to be extracted, such as for example an insoluble amine, advantageously then with an intermediate drying operation.

The following examples are intended to illustrate the invention without however limiting the scope thereof.

Examples 1, 2, 5, 10 and 12 were carried out according to the invention. Examples 3(C), 4(C), 6(C), 7(C), 8(C), 9(C), 11(C) and 13(C) were carried out by way of comparison.

### EXAMPLES 1 and 2

The catalytic system was prepared from amine PRIMENE 81-R, platinum (IV) chloride and tin (II) chloride and solvent SHELLSOL K.

The amine PRIMENE 81-R was a tent-alkyl primary amine, sold by Rohm and Haas. This is a mixture of amines, of which the number of carbon atoms was from 12 to 14. The solvent SHELLSOL K, sold by Shell, is composed of a mixture of hydrocarbons, mainly of aliphatic nature. The solvent used in these examples had an initial boiling point of 193° C. and a final boiling point of 245° C.

The amine PRIMENE 81-R was first mixed with the solvent SHELLSOL K in a 50/50 weight ratio. Added simultaneously and with stirring to 100 ml of this mixture were 0.76 g of platinum (IV) chloride, i.e. 22.6 mmol/l, and 0.43 g of tin (II) chloride, i.e. 22.6 mmol/l. The catalytic system was then prepared by saturating the solution with gaseous hydrogen chloride.

The reaction between acetylene and hydrogen chloride was carried out in the following manner:

A pyrex reactor having an internal volume of 45 ml, equipped with a double jacket in which a heat transfer oil circulated and a device for introducing reactants composed of a sintered glass nozzle intended to ensure the dispersion of the gases in the liquid medium, was charged with 30 ml of the solution prepared above.

The solution was heated either at 125° C. (Example 1), or at 150° C. (Example 2) and a gas flow containing a mixture of hydrogen chloride and acetylene with a HCl/C₂H₂ molar ratio of 1.16 was introduced into the reactor. The residence time of the gases in the reactor, that is to say the ratio of the reactor volume to the volume flow rate of the reactants at the reaction temperature was 5 s. The gaseous product exiting the reactor was analysed by gas chromatography. The only reaction products observed were vinyl chloride (VC) as the main product, accompanied by traces of 1-chloroprene (1CPr). The results are given in Table I. The amount of VC produced is expressed in moles of VC per hour and per mole of transition metal or in grams of VC per hour and per litre of catalytic system.

### EXAMPLES 3(C) and 4(C)

Examples 1 and 2 were reproduced without the addition of tin (II) chloride (identical amounts, by weight, of amine PRIMENE 81-R and of solvent SHELLSOL K and identical molar amount of platinum (IV) chloride).

The acetylene hydrochlorination reaction was carried out under the same conditions as in Examples 1 (Example 3(C)) and 2 (Example 4(C)). The results are given in Table I.

TABLE I

| | | | | VC produced | |
|---|---|---|---|---|---|
| Ex. | Amine/solvent | Residence | Temperature | (mol VC · h⁻¹ · mol | VC produced |
| No. | weight ratio | time (s) | (° C.) | metal⁻¹) | (g · h⁻¹ · 1⁻¹) |
| 1 | 50/50 | 5 | 125 | 21 | 29.7 |
| 2 | 50/50 | 5 | 150 | 170 | 240.1 |
| 3 (C) | 50/50 | 5 | 125 | 12 | 16.9 |
| 4 (C) | 50/50 | 5 | 150 | 139 | 196.3 |

It can be seen, from studying Table I, that the platinum (IV) chloride/tin (II) chloride pair (Examples 1 and 2) has made it possible to achieve a productivity that is significantly higher than that obtained with platinum (IV) chloride used alone (Examples 3(C) and 4(C)).

### EXAMPLES 5 to 9(C)

Example 2 was reproduced but using, as the catalytic system in place of the platinum (IV) chloride/tin (II) chloride pair, the platinum (II) chloride/triphenylphosphine oxide pair (Example 5), the platinum (II) chloride/triphenylphosphite pair (Example 6(C)), the platinum (II) chloride/triphenylphosphine pair (Example 7(C)), the platinum (II) chloride/tetramethylenediamine pair (Example 8(C)) or platinum (II) chloride alone (Example 9(C)). For each of these examples, the molar amount of these catalytic systems was equal to 22.6 mmol/l of platinum (II). The second optional compound of the pair was present at the same concentration.

The acetylene hydrochlorination reaction was carried out under the same conditions as in Example 2. The results are given in Table II.

TABLE II

| | | | | VC produced | |
|---|---|---|---|---|---|
| Ex. | Amine/solvent | Residence | Temperature | (mol VC · h⁻¹ · mol | VC produced |
| No. | weight ratio | time (s) | (° C.) | metal⁻¹) | (g · h⁻¹ · 1⁻¹) |
| 5 | 50/50 | 5 | 150 | 178 | 251.4 |
| 6 (C) | 50/50 | 5 | 150 | 40 | 56.5 |
| 7 (C) | 50/50 | 5 | 150 | 0 | 0 |
| 8 (C) | 50/50 | 5 | 150 | 66 | 93.2 |
| 9 (C) | 50/50 | 5 | 150 | 176 | 248.6 |

It can be seen, from studying Table II, that the platinum (II) chloride/triphenylphosphine oxide pair (Example 5) has made it possible to achieve a productivity that is significantly higher than that obtained with other pairs such as the platinum (II) chloride/triphenylphosphite pair (Example 6(C)), the platinum (II) chloride/triphenylphosphine pair (Example 7(C)) and the platinum (II) chloride/tetramethylenediamine pair (Example 8(C)).

### EXAMPLES 10 to 11(C)

Examples 5 and 9(C) were repeated in order to compare the effect of time on the productivity (Examples 10 and 11(C)).

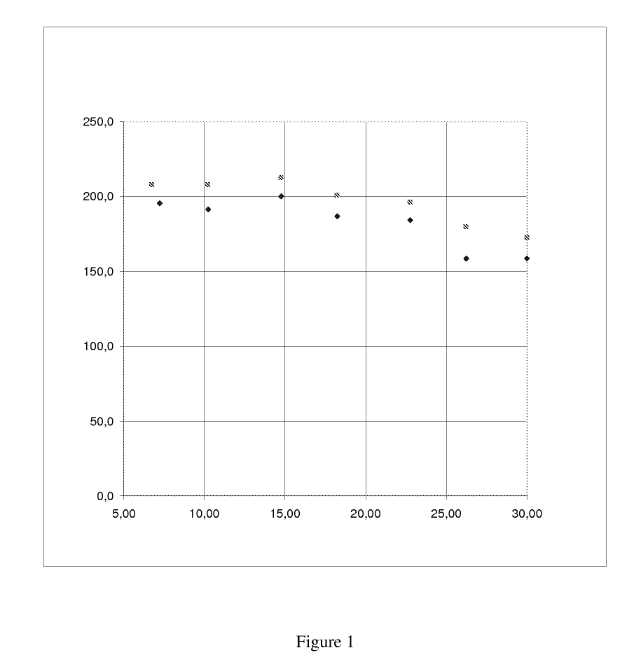

FIG. 1 illustrates, on the y-axis, the productivity (amount of VC produced in moles of VC per hour and per mole of transition metal) as a function of time, on the x-axis, expressed in hours, for Examples 10 (▪) and 11(C) (♦).

It can be seen, from studying FIG. 1, that the platinum (II) chloride/triphenylphosphine oxide pair (Example 10) has made it possible to achieve a productivity that remains higher over the first 30 hours than that obtained when platinum (II) chloride is used alone (Example 11(C)).

### EXAMPLES 12 to 13(C)

Example 2 was reproduced but using, as the catalytic system in place of the platinum (IV) chloride/tin (II) chloride pair, the palladium (II) chloride/triphenylphosphine pair (Example 12) in an amount of 22.6 mmol/l or palladium (II) chloride in an amount of 22.6 mmol/l (Example 13(C)).

The acetylene hydrochlorination reaction was carried out under the same conditions as in Example 2. The results are given in Table III.

TABLE III

| | | | | VC produced | |
|---|---|---|---|---|---|
| Ex. | Amine/solvent | Residence | Temperature | (mol VC · h⁻¹ · mol | VC produced |
| No. | weight ratio | time (s) | (° C.) | metal⁻¹) | (g · h⁻¹ · 1⁻¹) |
| 12 | 50/50 | 5 | 150 | 114 | 161 |
| 13 (C) | 50/50 | 5 | 150 | 69 | 97.5 |

It can be seen, from studying Table III, that the palladium (II) chloride/triphenylphosphine pair (Example 12) has made it possible to achieve a productivity that is significantly higher than that obtained with palladium (II) chloride used alone (Example 13(C)).

1- A catalytic hydrochlorination system comprising at least one amine hydrochloride and at least one group VIII metal compound selected from the group consisting of mixtures of a platinum (IV) compound with tin (II) chloride, mixtures of a platinum (II) compound with triphenylphosphine oxide, and mixtures of a palladium (II) compound with triphenylphosphine.

2. The catalytic system according to claim 1, wherein the group VIII metal compound is selected from the group consisting of the mixture of platinum (IV) chloride with tin (II) chloride, the mixture of platinum (II) chloride with triphenylphosphine oxide, and the mixture of palladium (II) chloride with triphenylphosphine.

3. The catalytic system according to claim 1, wherein the molar ratio of the tin (II) chloride, the triphenylphosphine oxide or the triphenylphosphine to the group VIII metal of the catalytic system is between 0.5 and 2.

4. The catalytic system according to claim 1, wherein the amine hydrochloride is chosen from the amine hydrochlorides of which the melting point is less than or equal to 25° C.

5. The catalytic system according to claim 4, wherein the amine hydrochloride corresponds to the formula: 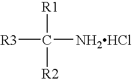
with R1 and R2 representing hydrogen atoms or identical or different alkyl or aryl groups, and R3 representing an alkyl or aryl group, said amine hydrochloride containing from 8 to 30 carbon atoms.

6- The catalytic system according to claim 4, wherein the content of group VIII metal compound expressed in millimoles per litre of amine hydrochloride is greater than or equal to about 1 mmol/l and less than or equal to about 1000 mmol/l.

7- The catalytic system according to claim 1, wherein the amine hydrochloride is chosen from the fatty amine hydrochlorides of which the melting point is greater than 25° C., and wherein the catalytic system comprises, in addition, an organic solvent.

8. The catalytic system according to claim 7, wherein the amine hydrochloride contains from 10 to 20 carbon atoms.

9- The catalytic system according to claim 7, wherein the weight ratio of the organic solvent to the fatty amine hydrochloride varies from about 0.1 to about 20, and wherein the content of group VIII metal compound expressed in millimoles per litre of catalytic system is greater than or equal to about 1 mmol/l and less than or equal to about 1000 mmol/l.

10- A process for manufacturing vinyl chloride by reaction of acetylene with hydrogen chloride in the presence of a catalytic system, wherein the catalytic system comprises at least one amine hydrochloride and at least one group VIII metal compound selected from the group consisting of mixtures of a platinum (IV) compound with tin (II) chloride, mixtures of a platinum (II) compound with triphenylphosphine oxide and mixtures of a palladium (II) compound with triphenylphosphine.

11. The process according to claim 10, wherein the amine hydrochloride is chosen from the amine hydrochlorides of which the melting point is less than or equal to 25° C.

12. The process according to claim 11, wherein the catalytic system is deposited on a solid support.

13. The process according to claim 11, wherein the catalytic system is used in the liquid phase.

14- The process according to claim 11, wherein the reaction is carried out at a temperature of about 40° C. to about 200° C.

15. The process according to claim 10, wherein the amine hydrochloride is chosen from the fatty amine hydrochlorides of which the melting point is greater than 25° C. and wherein the catalytic system comprises, in addition, an organic solvent.

16. The process according to claim 15, wherein the reaction is carried out at a temperature of about 40° C. to about 180° C.

17- The process according to claim 10, wherein the hydrogen chloride and the acetylene are used in a molar ratio of about 0.5 to about 3.
